(19) United States
(12) Patent Application Publication
(10) Pub. No.: US 2005/0204431 A1
(43) Pub. Date: Sep. 15, 2005

(54) Use of specific myb genes for the production of transgenic plants tolerant to biotic and abiotic stresses

(76) Inventors: Immacolata Coraggio, Roma (IT); Franca Locatelli, Roma (IT); Marcella Bracale, Varese (IT); Candida Vannini, Varese (IT)

Correspondence Address: NIXON & VANDERHYE, PC, 901 NORTH GLEBE ROAD, 11TH FLOOR, ARLINGTON, VA, 22203, US

(21) Appl. No.: 10/521,811

(22) PCT Filed: Jul. 21, 2003
(86) PCT No.: PCT/IB03/02878

§ 371 Date: Apr. 19, 2005

(30) Foreign Application Priority Data
Jul. 23, 2002 (IT) M12002A001624

(51) Int. Cl. A01H 1/00; C12N 15/82; C07H 21/04

(52) U.S. Cl. 800/289; 536/23.6; 530/370; 435/468

(57) ABSTRACT

The present invention relates to the use of Y11414 gene or its functional homologues for the production of plants tolerant to biotic and abiotic stresses, such as salt-induced stress, dehydration-induced stress, oxidative stress, and osmotic stress and the use of products which comprise said genes sequences, such as expression cassettes and biological vectors useful in the preparation of transgenic plants.

### SUBJECT OF THE INVENTION

The present invention relates to the use of particular genes of the Myb family for the production of plants that are capable of tolerating certain biotic and abiotic stresses, especially the use of certain Myb genes of the R2R3 class and of the proteins associated therewith that are implied in the defense of the plants against various adverse environmental conditions. The invention also relates to the use of products comprising the sequences of said genes, such as expression boxes (cassettes) and biological vectors that are useful in the preparation of transgenic plants.

### STATE OF THE ART

The plants are constantly subjected to the attack of enormous quantities of microorganisms, such as fungi, bacteria, viruses and of superior pathogenic organisms as well, against which they protect themselves by putting in operation defense mechanisms that are available in the plant itself. Such defense processes not always turn out to be enough for effectively fighting the pathogen, with consequent deleterious effects for the afflicted plant.

Besides the biotic stresses, the plants are subjected to environmental attacks of various type that cause modifications—sometimes relevant ones—of the environment the plant lives in. Thus, for example, cold, a high salinity or dehydration of the soil cause stress and damage to the plant.

The biotic and abiotic stresses are strongly limiting factors for the growth and development of the plants, and can be the cause of serious damages for the productivity of the species of interest, for the quality and nutritional value of the agricultural products, and the obtainment of better tolerant plants is an important objective in the public research programmes of numerous countries.

Said research has brought in the last years to the discovery and cataloguing of the genes that are activated as a response to the environmental stresses, both biotic and abiotic, into two large classes, especially the class of genes coding for regulator proteins that are implicated in the perception, transduction and amplification of the stress signal that activate and modulate the expression of genes that are directly involved in the acquisition of the tolerance (Zhu, J. K., Hasegawa, P. M. and Bressan, R. 1997, Critical review in Plant Sci. 16:253; Gu, Y. Q., Wildermuth, M. C., Chakravarthy, S., Lho, Y. T., Yang, C., He, X., Han, Y. and Martin, G. B. 2002, Plant Cell, 14, 817) and the class of genes which perform a direct protection/shelter function on fundamental biological processes, the expression of which is at the basis of the biochemical and physiological response and, consequently, of the tolerance to stresses (Thomashow, M. F. 1999, Annu. Rev. Plant Physiol. Plant Mol. Biol., 50:571; Schenk, P. M., Kazan, K., Wilson, I., Anderson, J. P., Richmond, T., Somerville, S. C. and Manners, J. M. 2000 Proc. Natl. Acad. Sci. USA, 97, 11655).

Transgenic plants which overexpress genes of this last class have highlighted that one single gene of this assembly only contributes very partially and marginally to the acquisition of the tolerance to environmental stresses, whilst plants which overexpress genes coding for transcriptional factors of the first class, said factors are capable of controlling and modulating the concurrent coordinated expression of several down-stream genes that are involved in the acquisition of the tolerance, exhibit better performance in inductive situations, as compared with non transformed, “wild type” plants, because a transformed plant with only a single transcriptional factor behaves like a plant that has been transformed with the full battery of genes it regulates (Jaglo-Ottosen, K. R., Gilmour, S. J., Zarka, D. G., Schbenberger, O. and Thomashow, M. F. 1998, Science, 28:104; Liu, Q., Kasuga, M., Sakuma, Y., Abe H., Miura, S., Yamaguchi-Shinozaki, K. and Shinozaki, K. 1998, Plant Cell, 10:1391; Schenk, P. M., Kazan, K., Wilson, I., Anderson, J. P., Richmond, T., Somerville, S. C. and Manners, J. M. 2000 Proc. Natl. Acad. Sci. USA, 97, 11655).

The present inventors have recently isolated cDNA clones of rice (Oryza sativa) that code for transcriptional factors of the Myb type and have demonstrated the function of certain of them in the response to stress. In the vegetable organisms, the Myb-like family of transcriptional factors is especially interesting because of its involvement in control and regulation of several vegetable cellular processes, such as the cellular proliferation and morfogenesis, the cellular metabolism, the response to stress.

As is well known, the sequences of the Myb genes are characterized by the presence of an N-terminal conserved region, which is followed by a region of variable length and sequence. The conserved region has the function of recognising and binding specific sequences in the promoters of the target genes and consists of a block comprising about 56 amino acids, characterized by tryptophans in a fixed position (triptophan domain). According to the number and type of triptophan domain that are present, the Myb genes are said to be of the R1R2R3, R1/R2, R2R3 type. The variable C-terminal region is usually charged with the transcriptional activities, with the cellular localization, with the post-transcriptional regulation and with the interaction with other proteins. The sequence homology in this region in Myb genes of different organisms is an evidence of the functional homology.

In particular, the inventors have isolated from rice a particular cDNA coding for a Myb factor of the class R2R3, the sequence of which has been deposited with the accession number N. Y11414 (EMBL). The Y11414 gene is constitutionally expressed at low levels in rice coleoptiles under optimum temperature conditions, its expression is strongly induced by low temperature treatments, 10° C., which is a sublethal temperature for the rice. The genes that are induced under this condition are considered to be important for the stress-protection under the most extremely cold temperatures. Both in heterologous systems (tobacco protoplasts) and in homologous systems (rice callus), Y11414 is capable of transactivating: 1) the promoter of the cold-inducible bean PAL gene, 2) the promoter of desaturase D9 of potato, which enzyme is cold-inducible and catalyses the formation of double bonds in the membrane fatty acids, this being one of the principal responses to the low temperatures. Transgenic Arabidopsis tholiana plants, both homozygous and single-insertion ones, which constitutively overexpress the Y11414 gene, exhibit an exceptional tolerance to treatments at down to −10° C. when compared with the “wild type” plants, thus demonstrating its real and effective capacity of imparting transgenic Arabidopsis tholiona plants tolerance to cold and freeze stresses (Osnato M. et al., Proceedings of the XLV Italian Society of Agricultural Genetics—SIGA Annual Congress Salsomaggiore Terme, Italy—26/29 Sep. 2001; Pandolfi et al., Plant Physiology 114, p 747. PGR97-079).

In particular, it has been demonstrated that the expression of the Y11414 gene, while it is induced by cold stress, is not actually induced by other environmental stresses, such as anoxia, high salinity, dehydration, nor is it induced by a hormone treatment with ABA (Pandolfi et al., supra).

### SUMMARY OF THE INVENTION

The inventors have now surprisingly discovered that the Y11414 gene and its functional homologues of other species impart tolerance to biotic and abiotic stresses, such as high salinity, dehydration, osmotic stress, oxidative stress, even though such genes are not directly induced in nature by these stresses.

Moreover, it has now been found that the plants which have been transformed with such genes show constitutive expression of various genes that are correlated to the tolerance to pathogens.

### DETAILED DESCRIPTION OF THE INVENTION

Thus, according to one of its aspects, the present invention relates to the use the Y11414 gene or its functional homologues thereof at other species for the production of transgenic plants tolerant to biotic stresses.

According to one of its aspects, the present invention relates to the use the Y11414 gene or its functional homologues thereof of other species for the production of transgenic plants tolerant to saline stress, dehydration stress, oxidative stress, and osmotic stress.

The grater tolerance to the above-described abiotic stresses is especially surprising if it is considered that, as indicated above, the Y11414 gene and the functional homologues thereof are not induced by such stresses in nature.

The term “genes”, as used in the present invention, is intended as an isolated polynucleotide sequence or isolated fragments of a polynucleotide sequence (DNA).

An “isolated polynucleotide sequence” is to be intended as being essentially devoid of the biological material it is normally associated with in natural products.

The genes according the present invention can be isolated from naturally available plants, of both the monocotyledonous or dicotyledonous types.

The term “biotic stress”, as used in the present invention, is intended as adverse environmental conditions caused by the attack of pathogenic organisms, like fungi, bacteria, viruses and other superior pathogens.

The term “transgenic plants tolerant to biotic and abiotic stresses”, as used in the present invention, is intended as plants that have been genetically modified and exhibit a greater adaptation and survival capacity in front of biotic and abiotic stresses as compared with the correspondent “wild type” plants.

The term “functional homologues”, as used in the present invention, is intended as the genes and the polynucleotide sequences that exert in the plants a function that is analogous to that exerted by the Y11414 gene in the rice plant. Preferably, said homologues are polynucleotide sequences that exhibit a sequence homology of at least 70% with the variable region of the Y11414 gene, advantageously of at least 80%, e.g. of 90%.

According to another of its aspects, the object of this invention is the use of polynucleotide sequences that exhibit a sequence homology of at least 70%, advantageously of at least 80%, for example of 90%, with the variable region of the Y11414 gene for the production of transgenic plants tolerant to the described stresses.

According to another of its aspects, the present invention relates to the use of the Y11414 gene, or of its functional homologues thereof of other species, for prevention and/or the treatment of the biotic stresses and of the damage caused by high salinity, dehydration, oxidative stress, and osmotic stress in plants.

The present invention also relates to the use of the functional variants, of the complementary sequences, and of the transcription products of the Y11414 gene, or of the functional homologues thereof, for the production of transgenic plants tolerant to the biotic stresses and to the stresses caused by high salinity, dehydration, oxidative stress, and osmotic stress.

An advantageous gene for the use according to the invention is the Y11414 gene itself.

The present invention also comprises the polypeptides that are coded by the Y11414 gene, by its functional homologues thereof of other species, by its functional variants or by the polynucleotide sequences that exhibit a sequence homology of at least 70%, advantageously of at least 80% or 90%, with the variable region of the Y11414 gene, and the use of said polypeptides according to the invention.

The functional homologues of other species of the Y11414 gene, and, advantageously, the polynucleotide sequences that exhibit a sequence homology of at least 70%, advantageously of at least 80%, for example 90%, with the variable region of the Y11414 gene, with the exclusion of the Y11414 gene itself, are part of the present invention.

Additionally, also the expression boxes (cassettes), the biological vectors, the host cells and the transgenic plants that comprise said functional homologues of the Y11414 gene, advantageously comprising the polynucleotide sequences that exhibit a sequence homology of at least 70% with the Y11414 gene, with the exclusion of the Y11414 gene itself.

For the use according to the invention, the selected gene is inserted into a “wild type” plant (or optionally an already transformed one) through the conventional gene technology procedures.

Illustratively of the agronomically interesting plants that can be transformed for the use according to the invention, there can be cited cereals (such as rice, maize and durum wheat), fruits and vegetables (such as tomato, potato, apple and other fruit trees), legumes (such as bean, pea), ornamental plants, but also other plants can be transformed according to the invention in order to confer them a greater resistance to stresses.

For that purpose, for example, the cDNA of the selected gene is operatively linked to a suitable promoter, and the thus obtained expression cassette is inserted into a biological vector, which in turn is inserted into the cells of the plants to be transformed.

Examples of suitable promoters are described e.g. in Osnato et al. (supra), where the use of the constitutional promoter CaMV35S for the dicotyledonous is described.

Other suitable promoters are for example Ubi1, which is constitutional for the monocotyledonous (Christen and Quail, Transgenic Research, 5, 213-218, 1996), or also Cor15 (Baker et al 1994 Plant Mol. Biol. 24:701-713).

The invention also relates to a method for the treatment and/or prevention of the damages caused by biotic, salt, dehydration, oxidative and osmotic stresses in the plants, said method comprising: inserting into said plants host cells comprising a polynucleotide sequence selected from the Y11414 gene, its functional homologues thereof in other species, and the polynucleotide sequences that exhibit a sequence homology of at least 70%, advantageously of at least 80% or 90%, with the Y11414 gene.

In order to verify the effects of the expression of the representative genes for the use according to the invention onto the protection of plants from stresses, the phenotypic tolerance effects, and, with a “microarray” analysis, the variations of the transcript induced in transgenic Arabidopsis plants by the overexpression of the rice transcriptional factor Y11414 have been assessed.

The experiments that have been conducted for demonstrative and illustrative purpose are presented in the experimental section hereunder, and are not to be construed as limiting in any way.

### EXPERIMENTAL SECTION

Preparation of Transgenic Plants

The cDNA of the Y11414 gene was put under the CaMV35S promoter and upstream of the terminator of gene Nos, the thus obtained expression cassette was inserted into the binary vector (E. coli—agrobacterium) PGA470. The latter was introduced by electroporation into the GV3101 strain of Agrobacterium tumefaciens, which was then used for transforming Arabidopsis thaliana (cv Wassilewskija) plants with the “floral dip” method.

Results

The computer-based analysis of the results obtained has pointed to the constitutive expression of several genes that are considered as being of a vital importance in the defense of the plant against pathogens, especially those involved in the defense response event known as SAR (Systemic Acquired Response). In particular, it turns out that many, or even all, of the genes that are involved in the biosynthesis of phenyl propanoids and lignins (dehydrokinase shikimato dehydrogenase, cinnamato-4-hydroxylase, PAL, cytochrome P450, EPSP, caffeoyl-CoA methyltransferase, cinnamoyl CoA reductase) are induced. The phenol compounds benzoic acid (BA) and salicylic acid (SA) are accumulated in high concentrations upon microbial attack, and are considered to be important mediators of the defense response. Both BA ad SA, as well as stilbene and other phytoalexines, are derivatives of the metabolism of the phenyl propanoids.

There has also been ascertained that also the transcription of genes coding for various types of PR (pathogen related), such as certain types of “hydroxyproline rich glycoproteins” (HRGPs, extensines), proteinase inhibitors, peroxidases, glutathion S-transferase and “lipid transfer protein” is induced.

Finally, it has turned out that there is induced the transcription of both ethylene-induced genes and at least one transcriptional factor that activates said genes. The role of ethylene combined with methyl jasmonate in the defense response of the plant is well known.

From the microarray analysis it has been found that also the expression of several genes coding for enzymes involved in the detoxifying action against active oxygen species (catalase, glutathion S-transferase, peroxidase) is induced.

Based on such findings, we have retained that it would be suitable to check the effect of the Y11414 gene on the tolerance to biotic and abiotic stresses.

Tolerance to Biotic Stresses

The level of resistance to pathogens has been assessed by mechanical inoculation of a virus (TNV, tobacco necrosis virus), a bacterium (Pseudomonas syringae pv. tomato) and a fungus (Botrytis cinerea), respectively, and the development of the infection was followed daily for 15 days. Upon termination of the development of the symptoms, the infection degree was assessed by means of computerized analysis of the infected leaf tissue surface and/or by counting the number of lesions in the case of TNV. For all of the three types of pathogens tested, the plants expressing Y11414 exhibit a high resistance level as compared with the wild type.

Tolerance to Abiotic Stresses

Tolerance to Dehydration Stress

The transgenic plants that had been transformed with Y11414 were also subjected to water withdrawal. Especially, the condition of “wild type” plants and transformed plants that have been deprived of irrigation for 10, 20 and 30 days have been observed. At ten and twenty days, the “wild type” plants show serious signs of chlorosis and dehydration, whilst the transformed plants do not appear to be damaged. At 30 days the “wild type” plants are completely dry, whilst the transformed ones, though they show damages, remain viable.

Tolerance to Salt Stress

The plants that had been transformed with Y11414 have proved better tolerant to salt stress, as demonstrated by treatments with 300 mM NaCl for one and two weeks, with a survival increase of from 12 (“wild type”) to 29% (transformed with Y11414) and of from 10 (“wild type”) to 27% (transformed ones) or with one-hour treatments with 600 mM NaCl, with a survival increase of from 20 to 60%.

Tolerance to Oxidative Stress

The tolerance to oxidative stress has been assessed by subjecting plants of the wild type and transformed with Y11414 to UV light treatments and ozone fumigations. Under both treatments, the transformed plants turn out to be extremely tolerant at doses that cause a high cell mortality in the wild type for both treatments.

From the above, and especially from the results of the experimentations that have been carried out, there comes out clearly the fundamental role performed by the above-described genes on the protection of plants against pathogens and from abiotic stresses like high salinity, osmotic stress, oxidative stress and dehydration, and consequently the importance the present invention has, especially in the agronomical field.

1. A method of production of a transgenic plant, said method comprising: transforming a plant with a Y11414 gene or a functional homologue thereof in other species to produce a transgenic plant which is stress tolerant.

2. The method according to claim 1 for prevention and/or treatment of biotic, salt-induced, dehydration-induced, oxidative, and osmotic stress.

3. The method according to claim 1, in which said gene is the Y11414 gene, its functional variants, complementary sequences, and transcription products thereof.

4. The method according to claim 1, in which said functional homologue is a polynucleotide sequence that exhibits a sequence homology of at least 70% with the variable region of the Y11414 gene.

5. A polynucleotide sequence characterized by a homology of at least 70% with the variable region of the Y11414 gene.

6. A polypeptide that is coded by the Y11414 gene, by a functional homologue thereof in other species, or by a polynucleotide sequence according to claim 5.

7. The use of a polypeptide according to claim 6 for the prevention and/or treatment of biotic, salt-induced, dehydration-induced, oxidative, and osmotic stress.

8. A method of production of a transgenic plant, said method comprising: transforming a plant with an expression cassette and/or a biological vector containing a Y11414 gene, a functional homologue thereof in other species, or a polynucleotide sequence according to claim 5 to produce a transgenic plant which is stress tolerant.

9. An expression cassette comprising a promoter operatively linked to a polynucleotide sequence according to claim 5.

10. A biological vector comprising a polynucleotide sequence according to claim 5 or an expression cassette comprising a promoter operatively linked to said polynucleotide sequence.

11. A vegetable host cell, transformed with the biological vector according to claim 10.

12. A transgenic plant comprising vegetable host cells according to claim 11.

13. A method for the treatment and/or prevention of the damages caused by biotic, salt, dehydration, oxidative and osmotic stresses in the plants, said method comprising transforming said plants with host cells comprising the Y11414 gene.

14. A method for the treatment and/or prevention of the damages caused by salt, dehydration, oxidative and osmotic stresses in the plants, said method comprising transforming said plants with host cells according to claim 11.

15. A method for the preparation of transgenic plants that are tolerant to the biotic, salt-induced, dehydration-induced, oxidative, and osmotic stress, said method comprising using the Y11414 gene, a functional homologue thereof, or a polynucleotide sequence according to claim 5.
